(19) United States
(12) Patent Application Publication
(10) Pub. No.: US 2015/0272650 A1
(43) Pub. Date: Oct. 1, 2015

(54) PUNCH INSTRUMENT

(71) Applicant: Alphatec Spine, Inc., Carlsbad, CA (US)

(72) Inventor: Nathanial Dubois, San Marcos, CA (US)

(21) Appl. No.: 14/673,918

(22) Filed: Mar. 31, 2015

Related U.S. Application Data
(60) Provisional application No. 61/973,373, filed on Apr. 1, 2014.

(51) Int. Cl. A61B 17/92; A61F 2/46; A61B 17/70

(52) U.S. Cl. CPC A61B 17/92; A61B 17/7074; A61F 2/4611; A61F 2/4603

(57) ABSTRACT

An instrument for preparing bone to receive an implant device is provided. The instrument includes a handle configured to be gripped by a user, a drive shaft and a support. The drive shaft extends axially from the handle. The support is configured to hold a spacer. The spacer is dimensioned in the same shape as the desired implant device. A piercing member is disposed on a distal end of the drive shaft. The drive shaft may be translated axially with respect to the spacer so as to slide the piercing member along the ramp-shaped member. The ramp-shaped member drives the piercing member beyond an outer surface of the spacer and into the bone. A stop may be mounted to the support. The stop is configured to limit the depth at which the spacer is inserted into the body.

### CROSS-REFERENCE TO RELATED APPLICATIONS

This application claims priority of U.S. Provisional Application 61/973,373 filed Apr. 1, 2014, the contents of which are incorporated herein by reference.

### FIELD OF THE INVENTION

The invention is directed to an instrument for preparing bone to receive an implant device, and in particular to an instrument that prepares and introduces one or more anchors into the bone so as to optimize the use of the implant device.

### BACKGROUND OF THE INVENTION

Implant devices having an anchor configured to fix the implant device within the body are currently known and used. The anchor is pointed and is configured to pierce a bone. It should be appreciated that a user installing the implant device may have a plurality of anchors, each of different dimensions, to choose from. The selection of the anchor may be based upon, inter alia, the size, physical health, or the density of the bone being treated.

Such implant devices include a cage bounding a space configured to hold biomaterial such as bone graft. It should be appreciated that the user may have a plurality of cages, each of different dimensions, to choose from. The selection of the cage may be based upon, inter alia, the size or spatial dimension of the space between adjacent bones in which the implant device is to be inserted. The implant device may be further configured to stabilize a pair of adjacent bones together.

An exemplary illustration of such an implant device is provided in FIG. 1. The implant device in FIG. 1A may be used to stabilize a pair of adjacent vertebrae and to introduce bone graft so as to repair damaged vertebrae. The anchor is configured to pierce the vertebrae so as to secure the implant device between adjacent vertebrae as shown in FIG. 1C. However, in certain instances the vertebrae surface may be hardened, and it may be difficult for the anchor to pierce the bone.

Accordingly, it remains desirable to have an instrument configured to prepare the bone to receive the anchors. It further remains desirable to have an instrument which can introduce a spacer so as to ensure that the implant device is of a proper shape and dimension to fit between adjacent vertebrae. It further remains desirable to have an instrument configured to insert one of a plurality of differently dimensioned anchors so as to better prepare the bone for a desired anchor, and one of a plurality of differently dimensioned spacers so as to provide the user with a reference as to the proper fit of a selected implant device.

### SUMMARY OF THE INVENTION

An instrument for preparing bone to receive an implant device is provided. The instrument may be configured to introduce one of a plurality of differently dimensioned anchors so as to optimize the insertion of a selected anchor. The instrument may be further configured to introduce one of a plurality of differently dimensioned spacers to provide the user with a reference as to the proper fit of a selected implant device.

The instrument includes a handle configured to be gripped by a user, a drive shaft and a support. The drive shaft extends axially from the handle. The support has an elongated body and is mounted to the handle. The support is configured to hold a spacer that can be mounted to the distal end of the support. The spacer is dimensioned in the same shape as the implant device. The spacer includes a ramp-shaped member.

The instrument further includes a piercing member. The piercing member is disposed on a distal end of the drive shaft. The drive shaft may be translated axially with respect to the spacer so as to slide the piercing member along the ramp-shaped member, beyond an outer surface of the spacer and into the bone.

The instrument may further include a stop. The stop may be mounted to the support. The stop is adjustable along an axis generally parallel to the drive shaft. The stop is configured to limit the depth at which the spacer is inserted into the body.

In an illustrative embodiment of the support, the support is a housing having an elongated body bounding a space. The drive shaft is disposed within the housing. The drive shaft is recessed within the housing so as to position the end portion of the piercing member adjacent to the ramp-shaped surface.

In another embodiment of the instrument the support is an elongated cylindrical member. The support includes a plurality of guides. Each of the guides includes a ringed member. The drive shaft is slidably disposed through the ringed member of one set of guides. The ringed members are dimensioned so as to allow the drive shaft to slide along an axis generally parallel to that of the support. Another set of guides are configured to hold the stop.

The piercing member may be pivotally mounted to the distal end of the drive shaft, wherein the distal end of the piercing member may be engaged with a bottom surface of the ramp-shaped member of the spacer such that the body of the piercing member is angled relative to the drive shaft.

### BRIEF DESCRIPTION OF THE DRAWINGS

A better understanding of the present invention will be had upon reference to the following detailed description when read in conjunction with the accompanying drawings wherein like reference characters refer to like parts throughout the several views and in which:

FIG. 1A is a perspective view showing an implant device having bone graft material;

FIG. 1B is a top down view of an implant device showing the anchors deployed;

FIG. 1C is a perspective view of an implant device showing the anchors deployed between two adjacent vertebrae;

FIG. 2 is a perspective view of an instrument for preparing the bone for receiving an implant;

FIG. 3 is view of the instrument shown in FIG. 2 taken from the bottom;

FIG. 4 is an exploded view of the instrument shown in FIG. 2;

FIG. 5 is an isolated view of the spacer shown in FIG. 2;

FIG. 6 is a perspective view of another embodiment of an instrument for preparing the bone for receiving an implant;

FIG. 7 is a view of the instrument shown in FIG. 6 taken from the bottom;

FIG. 8 is an exploded view of the instrument shown in FIG. 6; and

FIG. 9 is an isolated view of the spacer shown in FIG. 6.

### DETAILED DESCRIPTION OF THE PREFERRED EMBODIMENTS

An instrument for preparing bone to receive an implant device is provided. The instrument may be configured to introduce one of a plurality of differently dimensioned anchors so as to optimize the insertion of a selected anchor. The instrument may be further configured to introduce one of a plurality of differently dimensioned spacers to provide the user with a reference as to the proper fit of a selected implant device.

The instrument includes a handle configured to be gripped by a user, a drive shaft and a support. The support is configured to hold a spacer that can be mounted to the distal end of the support. The spacer is dimensioned in the same shape as the implant device. The spacer includes a ramp-shaped member configured to help insert the spacer into the surgical sight.

The instrument further includes a piercing member. The piercing member is disposed on a distal end of the drive shaft. The drive shaft may be translated axially with respect to the spacer so as to slide the piercing member along the ramp-shaped member, beyond an outer surface of the spacer and into the bone. Thus, preparation of the surgical sight may be done by performing a simple thrust.

In another embodiment of the instrument the support is an elongated cylindrical member. The support includes a plurality of guides. Each of the guides includes a ringed member. The drive shaft is slidably disposed through the ringed member of one set of guides. The ringed members are dimensioned so as to allow the drive shaft to slide along an axis generally parallel to that of the support. Another set of guides are configured to hold the stop.

The piercing member may be pivotally mounted to the distal end of the drive shaft, wherein the distal end of the piercing member may be engaged with a bottom surface of the ramp-shaped member of the spacer such that the body of the piercing member is angled relative to the drive shaft. The piercing member is operatively connected to a head. The head drives the piercing member along the ramp-shaped member so as to piercing the bone and thus prepare adjacent bones for insertion of the implant device and installation and deployment of the anchors within the bone

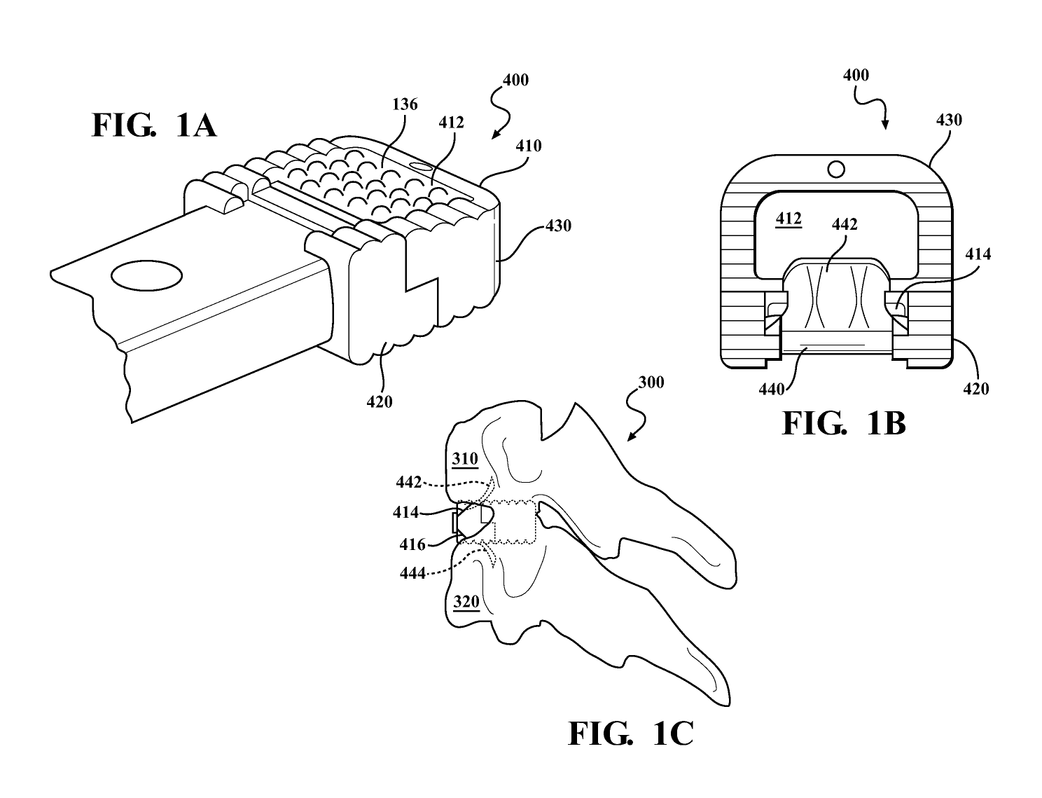

With reference now to FIG. 1, an exemplary embodiment of an implant device 400 is provided. The implant device 400 is configured to secure adjoining bones 310, 320 together and also to introduce a biomaterial so as to help heal damaged bones. Specifically, the implant device 400 provided in FIG. 1 is used to provide stability and introduce bone graft to adjoining vertebrae 310, 320.

The implant device 400 includes a cage 410. The cage 410 is formed of two parts 420, 430. The first part 420 is formed of a strong and durable material such as medical grade titanium or stainless steel. The second part 430 may be formed of a more resilient material such as polyetheretherketone, “PEEK.” The cage 410 includes an opening 412 configured to hold the biomaterial. For illustrative purposes the biomaterial shown is bone grafting BG material.

The implant device 400 further includes an anchor 440. The anchor 440 includes two piercing heads 442, 444 disposed opposite of each other and configured to ride over a respective top 414 and bottom edge 416 of the cage 410 so as to penetrate into the adjoining vertebrae.

It should be appreciated that the implant device 400 may be dimensioned in various sizes to accommodate the physical characteristics of a patient. Namely, the space between the vertebrae may be different from patient to patient. Accordingly, it is desirable to have a plurality of differently sized cages 410 so as to ensure a proper fitting with respect to the vertebrae.

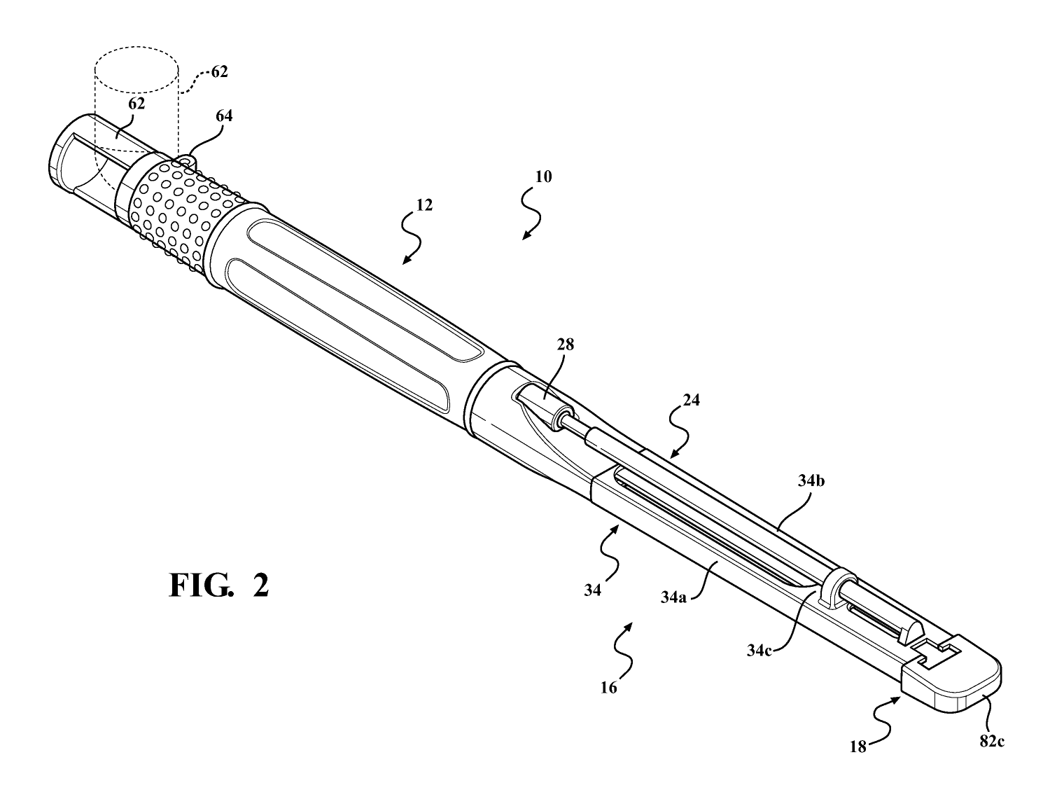

With reference now to FIGS. 2-5, an illustrative example of an instrument 10 for preparing a bone 300 to receive an implant device 400 is provided. The instrument 10 is configured to pierce adjacent bone structure 310, 320 so as to facilitate the insertion of the implant device 400 and also to provide a reference for determining if the size of the implant device 400 is proper with respect to adjoining bones 310, 320.

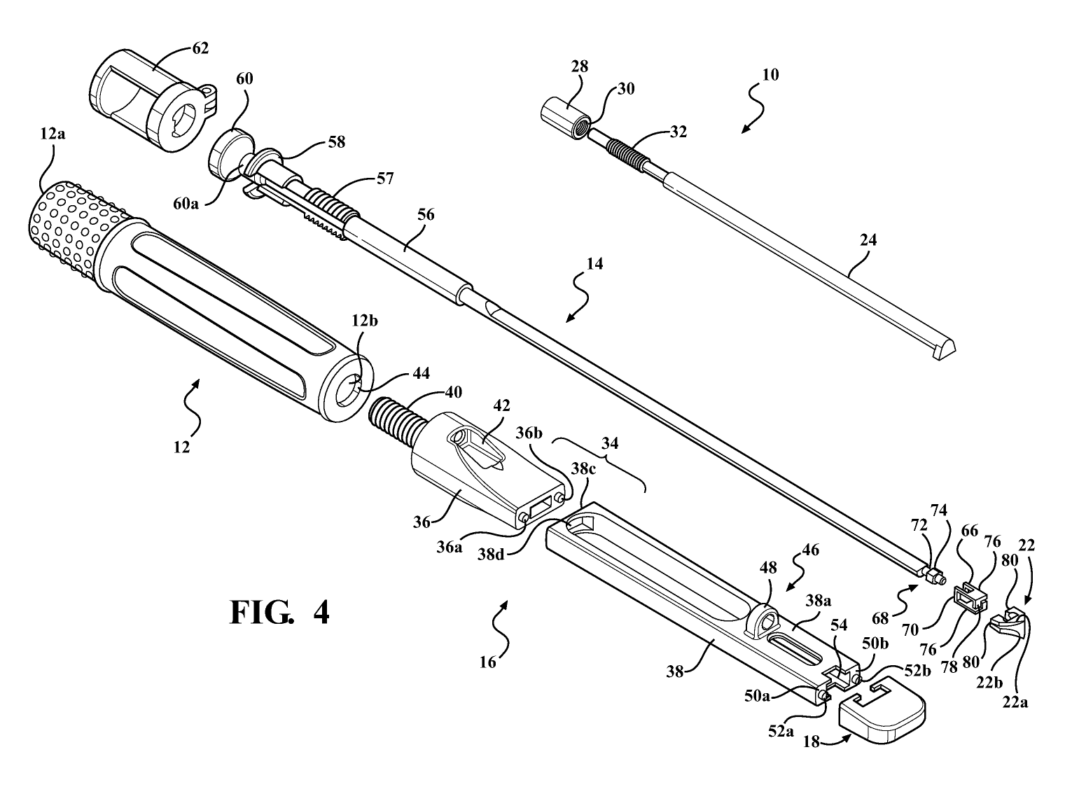

The instrument 10 has a longitudinal axis and includes a handle 12. The handle 12 is generally cylindrical so as to be gripped by a user. The handle 12 includes a through-hole so as to define a proximal and distal opening 12b. The distal opening 12b is shown in FIG. 4. A proximal end of the handle 12 may be lined with a resilient material to facilitate the grip of a user. The material may include features such as protrusions to further facilitate the grip of a user. As shown in FIG. 4, a portion of a drive shaft 14 is housed within the handle 12.

The drive shaft 14 extends axially from the handle 12. The drive shaft 14 is formed from a rigid material such as titanium or steel. The drive shaft 14 may be axially translated. The instrument 10 includes a support 16. The support 16 has an elongated body and is mounted to the handle 12.

The instrument 10 includes a spacer 18. The spacer 18 is mounted to a distal end of the support 16. It should be appreciated that throughout the drawings one spacer 18 is shown mounted to the instrument 10. However, various spacers 18 of different dimensions may be mounted to the instrument 10. The spacer 18 is dimensioned in the same shape as the intended implant device 400 so as to provide a reference for the user to determine if the selected implant device 400 fits properly.

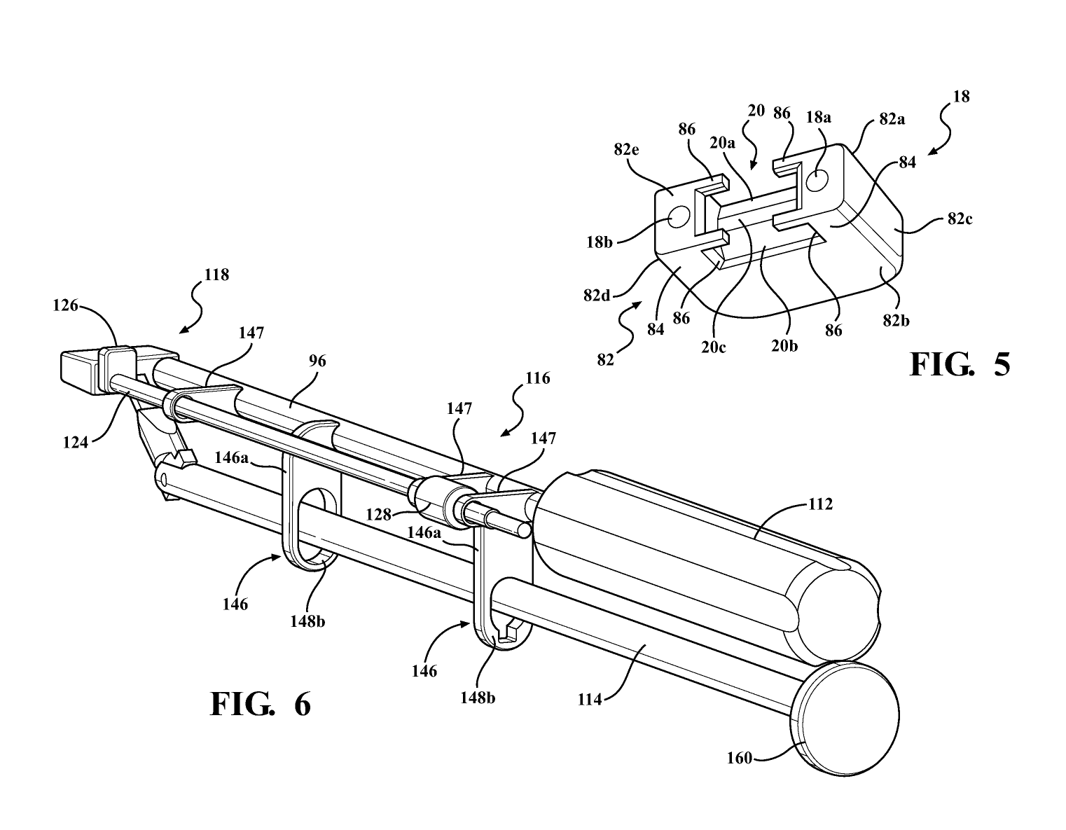

With reference now to FIG. 5, the spacer 18 includes a ramp-shaped member 20. The ramp-shaped member 20 includes at least one ramp surface 20a. The ramp surface 20a is angled obtusely when measured from the proximal end to the distal end of the instrument 10. Preferably, the spacer 18 includes two ramp surfaces 20a, 20b. The ramp surfaces 20a, 20b are joined at a front edge 20c.

The instrument 10 further includes a piercing member 22 (e.g., see FIGS. 2, 3, 6). The piercing member 22 is disposed on a distal end of the drive shaft 14. The drive shaft 14 is axially displaceable with respect to the spacer 18 and the piercing member 22 is configured to slide along the ramp-shaped member 20 so as to slide along the obtusely angled surface and be driven beyond an outer surface of the spacer 18 and into an adjoining bone similar to the anchor 440 shown in FIG. 1.

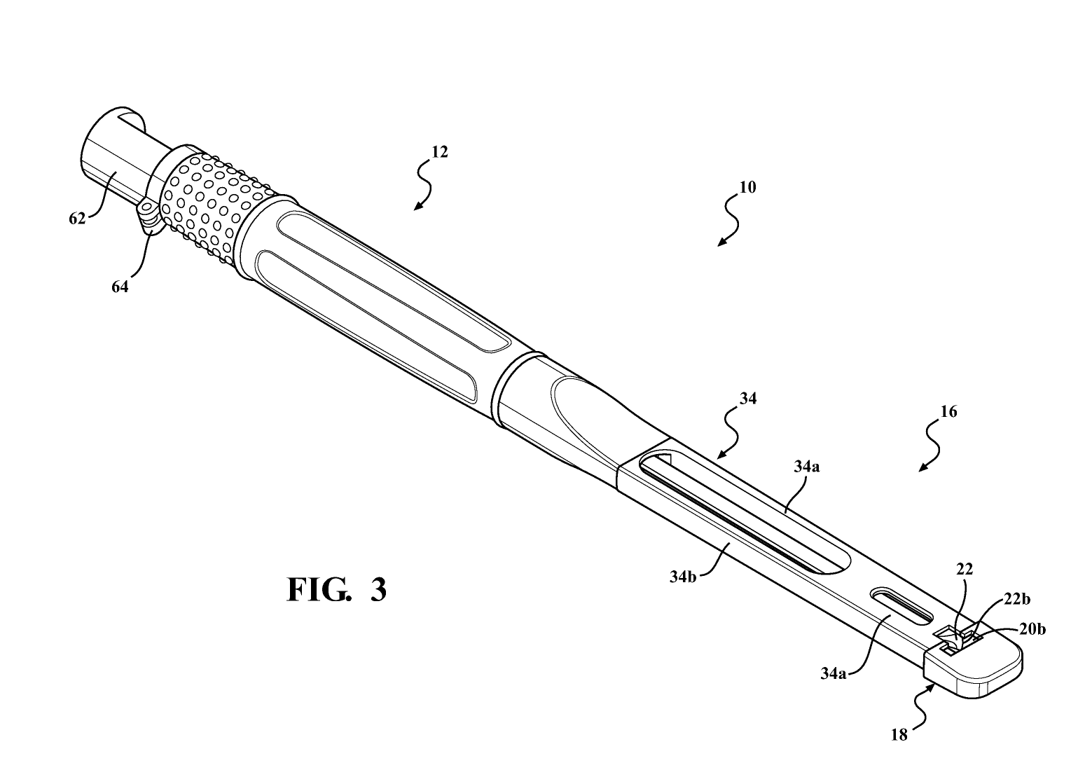

FIG. 4 provides an illustrative embodiment of a piercing member 22 wherein the piercing member 22 has a first head 22a and a second head 22b. The first and second heads 22a, 22b are split apart and each project outwardly from the axis of the drive shaft 14. A description of the piercing member 22 will be provided in conjunction with the spacer 18 shown in FIG. 5. The first and second heads 22a, 22b have a pointed end and are angled away from each other. The piercing member may be made of a durable and resilient material such as titanium, stainless steel, steel, etc. The first head 22a is configured to engage over the first ramp surface 20a. FIG. 3, shows second head 22b is displaced over the second ramp surface 20b of the ramp-shaped member 20. The edge of the ramp-shaped member 20 is disposed within a slot, which is axially aligned with the drive shaft 14. The slot is formed between the first head 22a and the second head 22b.

The instrument 10 further includes a stop 24. The stop 24 is mounted to the support 16 and is movable along an axis parallel to the drive shaft 14. The stop 24 is configured to limit the depth at which the spacer 18 is inserted into a body. The stop 24 includes a contact surface 26 formed at a distal end of the stop 24. The stop 24 is configured so as to axially displace the contact surface 26 with respect to the distal end of the instrument 10 so as to set the depth at which the spacer 18 and the piercing members 22 are deployed within the body. For illustrative purposes the stop 24 is shown moved axially by a collar 28 having a threaded bore 30. The collar 28 is rotatably mounted to a threaded end 32 of the stop 24. Thus, it should be appreciated that rotation of the collar 28 displaces the contact surface 26 of the stop 24 axially with respect to the length of the instrument 10.

With reference again to FIGS. 2-4, an exemplary embodiment of the instrument 10 is provided wherein the support 16 is a housing 34. The housing 34 has a pair of side walls 34a, 34b spaced apart from each other and extending between a top wall 34c and a bottom wall 34d so as to define a space. The stop 24 is mounted to a top wall 34c of the housing 34. The housing 34 extends axially from the handle 12 so as to form a unitary elongated structure.

FIG. 4 shows the housing 34 as having a first housing portion 36 and a second housing portion 38. The first housing portion 36 and the second housing portion 38 may be coupled together as shown in FIG. 3 or may be formed as a single unit. The first housing portion 36 has a threaded male member 40 disposed on the proximal end of the first housing portion 36. The distal end of the first housing portion 36 includes a pair of pegs 36a, 36b configured to fittingly engage a pair of holes (not shown) disposed on a proximal end of the second housing portion 38. The first housing portion 36 further includes a retaining portion 42. The retaining portion 42 is configured to hold the collar 28 of the stop 24. The male threaded member 40 may be threadably coupled to a threaded bore 44 disposed on a distal end of the handle 12.

The second housing portion 38 includes a back wall 38c having an opening 38d configured to slidingly fit the drive shaft 14. The top wall 38a of the second housing portion 38 includes a guide 46 having a ringed member 48 configured to slidingly fit a portion of the stop 24. The distal end of the second housing 38 is configured to engage the spacer 18. For illustrative purposes, the distal end of the second housing 38 includes two peripheral end walls 50a, 50b each of which are spaced apart from each other. End wall 50a has a peg 52a and end wall 50b has a peg 52b. Pegs 52a, 52b are configured to fittingly receive openings 18a, 18b, respectively, of the spacer 18 shown in FIG. 5. A distal edge of the second housing 38 is recessed with respect to the end walls 50a, 50b so as to form an opening 54 to accommodate the projection of piercing members 22.

With reference again to FIG. 4, the instrument 10 may further include a sleeve 56. The sleeve 56 includes a threaded outer surface 57 and a lip 58 extending radially from a proximal end of the sleeve 56 and is configured to fit within the proximal opening (not shown) generally centrally located at the proximal end of the handle 12. As shown in FIG. 4, the handle 12 includes a through bore 12b wherein the distal and proximal ends of the through bore 12b are threaded so as to threadingly receive the male member 40 of the first end of the housing 34 and the threaded outer surface 57 of the sleeve 56, respectively.

The drive shaft 14 is inserted into the sleeve 56. The drive shaft 14 includes a head portion 60. The drive shaft 14 is configured such that the head portion 60 extends beyond the proximal opening of the handle 12 wherein the remaining length of the drive shaft 14 is disposed within the body, through the through bore 12b, of the handle 12 and the space defined by the housing 34. The sleeve 56 is an elongated body configured to slidingly house a portion of the drive shaft 14 so as to allow the head portion 60 to drive the drive shaft 14 axially towards the distal end of the housing 34.

The instrument 10 may further include a cap 62 (e.g., see FIGS. 2-4). The cap 62 is mounted to a proximal end of the handle 12 and is movable between an open and closed position. The cap 62 covers the proximal end of the drive shaft 14 when in the closed position so as to prevent the head portion 60 from being displaced. Although the figures show the cap 62 having a hinge 64 so as to pivot the cap 62 outwardly to the open position as illustrated by the arrow in FIG. 3, it should be appreciated that the cap 62 may be removably attached to the proximal end of the handle 12 in other ways such as a screw top or a snap fit engagement. Cap 62 may further provide a surface to receive the impact of a hammer so as to allow the instrument to position the spacer 18 between adjacent vertebrae without deploying a piercing member 22.

With reference again to FIG. 4, the instrument 10 may further include an adaptor 66. The adaptor 66 is configured to mount to the distal end of the drive shaft 14 and hold the piercing member 22 to the distal end of the drive shaft 14. The adaptor 66 thus provides the instrument 10 with the ability to mount one of a plurality of differently dimensioned piercing members 22. Though the figures show one piercing member 22, it should be appreciated that multiple piercing members 22 of different sizes and shapes may be used so as to provide an optimized piercing signature within the bones 310, 320 of the patient. The adaptor 66 may be configured to mount to a nubbed portion 68 formed on the distal end of the drive shaft 14.

The adaptor 66 includes a pair of spaced apart windows 70. The windows 70 are resilient and have memory so as to be able to overcome the nub portion 68 and close onto a neck 72 of the drive shaft 14. The nub portion 68 includes a pair of planar side surfaces 74. The adaptor 66 may further include a pair of spaced apart side walls 76 dimensioned to fittingly engage and pinch therebetween the planar side surfaces 74 of the nub portion 68. A front wall 78 of the adaptor 66 may be configured to be enclosed by a pair of legs 80 formed on a back end of the piercing member 22.

The piercing member 22 may include a first head 22a and a second head 22b. The first and second heads 22a, 22b are angled outwardly with respect to each other wherein the distal ends may slightly curve outwardly. The drive shaft 14 has a length such that the distal ends of the heads 22a, 22b of the piercing member 22 may be axially driven against a front edge of the ramp-shaped member 20 and ramp surfaces 20a, 20b of the spacer 18 when the spacer 18 is attached to the housing 34.

The head 60 of the drive shaft 14 includes a neck 60a having an outer surface dimensioned to slidingly fit within the inner wall surface of the sleeve 56 wherein a predetermined amount of force must be applied to the head 60 so as to drive the neck 60a into the sleeve 56 thus driving the piercing member 22 into engagement with the ramp-shaped member 20 of the spacer 18.

With reference now to FIG. 5, an illustrative embodiment of the spacer 18 for use with the instrument 10 is provided. The spacer 18 includes a body 82 having a top surface 82a, a bottom surface 82b, a pair of side walls 82c, 82d, and a front surface 82e (see FIG. 2). The top and bottom surfaces 82a, 82b are contoured at the front edges. The spacer 18 includes a plurality of legs 84 extending away from the ramp-shaped member 20. The legs 84 are spaced apart from each other and each include a bore 18a, 18b configured to receive the pegs 52a, 52b formed at the distal end of the housing 34.

The ramp-shaped member 20 is recessed with respect to the distal ends of the legs 84. Each of the legs 84 further includes a pair of prongs 86. Each of the prongs 86 extends inwardly along an axis. The legs 84 extend outwardly with respect to the ramp-shaped member 20 so as to form an opening with respect to the distal end of the spacer 18 for which the piercing members 22 may slide through and into the respective bones of the patient.

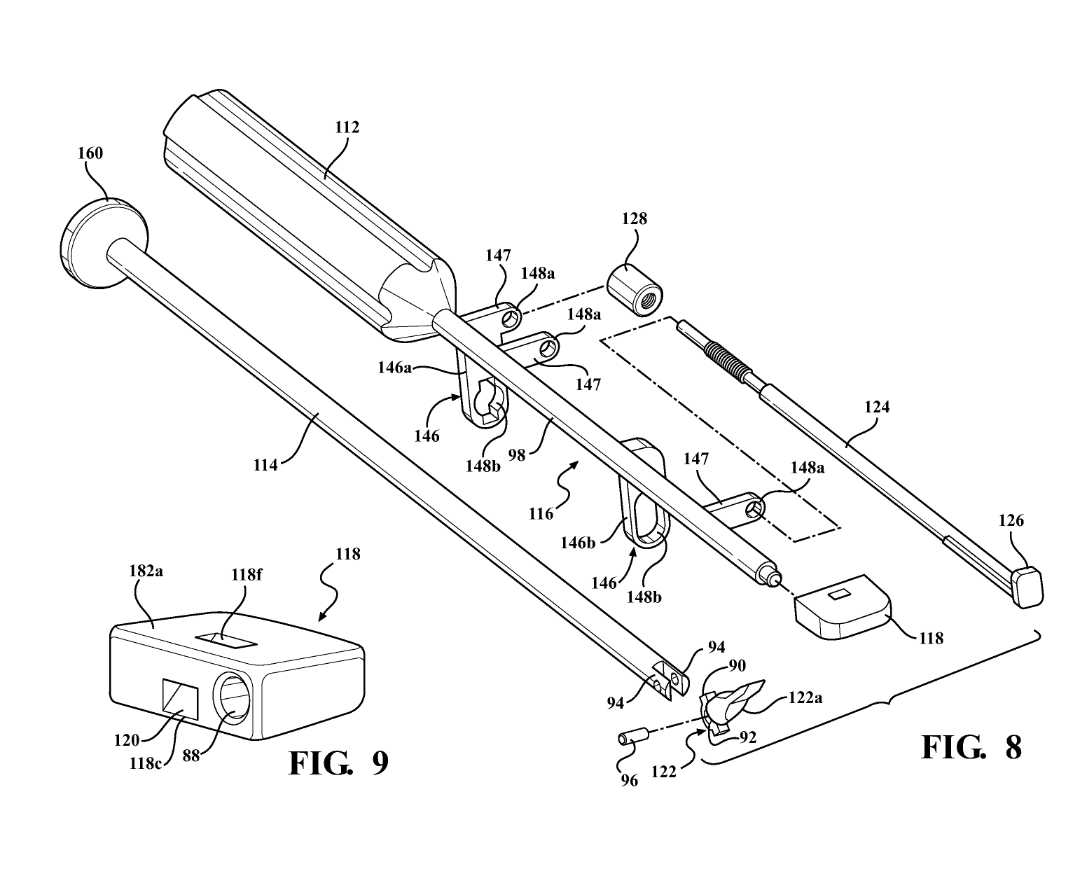

With reference now to FIGS. 6-9, another illustrative example of an instrument 110 with a longitudinal axis is provided wherein like parts are referenced by like numerals increased by 100. As shown in the figures, the support 116 is an elongated member 98 extending from the center axis of the handle 112. The support 116 may be formed of a durable and rigid material such as steel, stainless steel, titanium and the like. The distal end of the support 116 may be threaded or as shown in FIG. 8 may be configured to form a snap fit engagement with the spacer 118. The elongated support 116 is fixedly mounted to the handle 112 and includes a plurality of guides. One set of guides 146 is configured to slidingly hold the stop 124 and yet another set of guides 147 is configured to slidingly hold the drive shaft 114.

As shown in FIG. 8, a pair of guides referenced herein as drive shaft guides 146 extend along a common axis. A proximal shaft guide 146a is adjacent the handle 112 and a distal shaft guide 146b is adjacent the distal end of the support 116. Each of the guides 146a and 146b includes an oblong ringed member 148b. Another pair of guides 147 extends outwardly and generally orthogonal to the drive shaft guides 146. Guides 147 are configured to slidingly hold the stop 124. Guides 147 are also referenced herein as stop guides 147. Each stop guide 147 includes a ringed member 148a having a generally circular opening. The proximal drive shaft guide 146a includes a ringed element 148a having an opening coaxial with the ringed member 148a of the stop guides 147. A collar 128 is rotatably mounted to the shaft of the stop 124 so as to axially displace the contact surface 126 with respect to the distal end of the support 116. The drive shaft 114 has a distal end configured to mount a piercing member 122.

With reference now to FIG. 9, an illustrative example of a spacer 118 for use with the instrument 110 shown in FIGS. 6-8 is provided. The spacer 118 includes a peripheral side opening 188 extending axially along the length of a side of the spacer 118. The ramp-shaped member 120 is centrally located and includes an edge 118c disposed on a bottom side surface of the front face of the spacer 118. A ramp surface 118f at a top surface 182a is angled upwardly with respect to the bottom edge 118c. The ramp surface 118f defines an opening angled within the body of the spacer 118.

The piercing member 122 may include a piercing body 122a and an anchor 90 having an eyelet 92 configured to fit between a pair of legs 94 formed on a distal end of the drive shaft 114. The legs 94 have an opening coaxial with each other. A pin 96 is configured to fit between the openings within legs 94 and the eyelet 92 of the anchor 90 so as to pivotably retain the piercing member 122 therein.

It should be appreciated that the spacer 118 shown in FIG. 9 is illustrative and not limiting to the scope of the invention and that the piercing member 122 may be modified to accommodate other shapes. For instance, the spacer 118 may include two ramp surfaces similar to that shown in FIG. 5 wherein in such an embodiment the piercing member 122 may have a pair of heads angled outwardly from each other forming a central slit wherein the opposing heads are configured to engage respective surfaces of the ramp-shaped member 120 similar to that shown in FIG. 2 and also in FIG. 3.

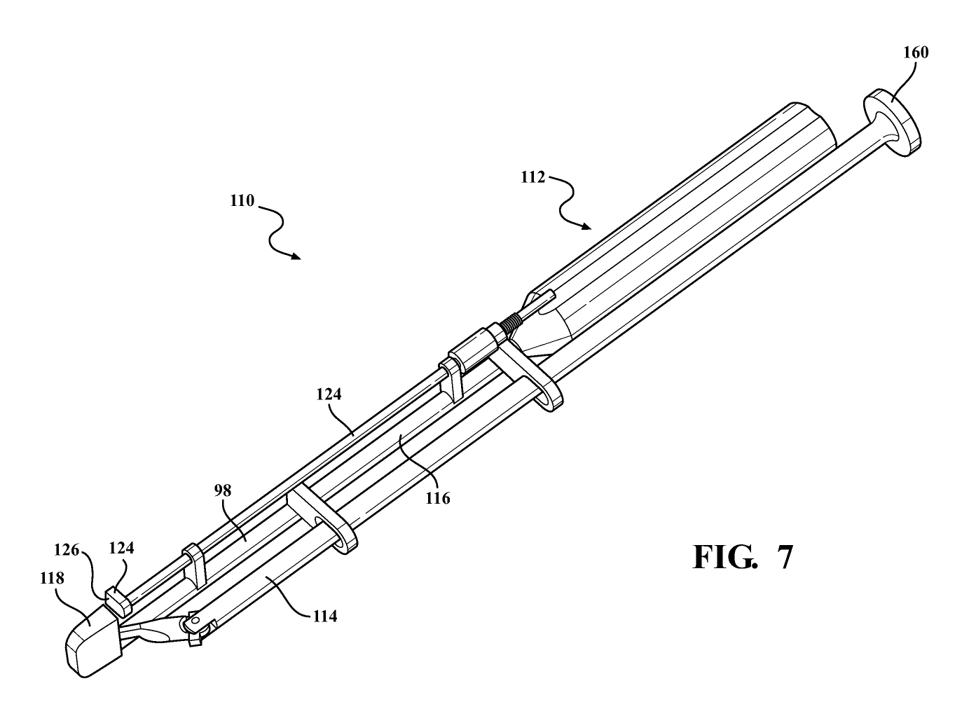

As shown in FIG. 7 the instrument 110 may be oriented such that the drive shaft 114 is disposed beneath the elongated support 116. The stop 124 is set so that the contact surface 126 is spaced apart from a distal end of the support 116 so as to place the spacer 118 in a predetermined and predesired depth within the body. Also, plunging the head of the drive shaft 114 forward drives the piercing member 122 upwardly so as to extend beyond the top surface of the spacer 118 and drive the forward element of the piercing member 122 into the bone. The instrument 110 may then be extracted and flipped over such that the drive shaft 114 is disposed above the support 116 wherein, actuation of the head 160 drives the piercing member 122 along the ramp-shaped member 120 downwardly so as to create a piercing within the bone and thus prepare adjacent bones for insertion of the implant device 400 and installation and deployment of the anchors 440 within the bone. Accordingly, the user may be able to determine the spacing and the dimensions of the spacer 118 with respect to the space in between a top and bottom adjacent vertebrae to ensure that the implant device 400, which is similarly dimensioned, is a properly fit.

The invention has been described in an illustrative manner. It is therefore to be understood that the terminology used is intended to be in the nature of words of description rather than limitation. Many modifications and variations of the invention are possible in light of the above teachings without varying from the scope and spirit of the invention described herein. As such, it is the claims, and all equivalents thereof, that define the scope of the invention.

1. An instrument for preparing a bone to receive an implant device, the instrument comprising:
a handle;
a drive shaft;
a support having an elongated body, the support mounted to the handle;
a spacer mounted to a distal end of the support, the spacer dimensioned in the same shape as the implant, the spacer having a ramp-shaped member; and
a piercing member disposed on a distal end of the drive shaft, the drive shaft longitudinally displaceable with respect to the spacer, the piercing member configured to slide along the ramp-shaped member of the spacer so as to be driven beyond an outer surface of the spacer.

2. The instrument as set forth in claim 1, further including a stop, the stop mounted to the support and movable along an axis parallel to the drive shaft, wherein the stop is configured to limit the depth at which the spacer is inserted into a body.

3. The instrument as set forth in claim 2, wherein the support is a housing bounding a space, the drive shaft disposed within the housing and the piercing member is registered to slide along the ramp-shaped member.

4. The instrument as set forth in claim 3, further including a sleeve mounted to the handle, wherein the sleeve is an elongated body configured to slidably house a portion of the drive shaft.

5. The instrument as set forth in claim 3, further including a cap, the cap mounted to a proximal end of the handle and movable between an open and closed position, wherein the cap covers a proximal end of the drive shaft when in the closed position.

6. The instrument as set forth in claim 3, further including an adaptor, the adaptor configured to mount to the distal end of the drive shaft, wherein the adaptor is configured to hold the piercing member to the distal end of the drive shaft.

7. The instrument as set forth in claim 3, wherein the piercing member includes a first head and a second head, the first and second head are angled outwardly with respect to each other.

8. The instrument as set forth in claim 2, wherein the support is an elongated member extending axially from the handle and having a plurality of guides.

9. The instrument as set forth in claim 8, wherein the at least one of the plurality of guides is two guides, and each of the plurality of guides includes a ringed member and the drive shaft is slidingly disposed within the two guides and generally parallel to the support.

10. The instrument as set forth in claim 9, wherein the piercing member is pivotably mounted to the distal end of the drive shaft, and the end portion of the piercing member is registered to the ramp-shaped member of the spacer.

11. An instrument for preparing a bone to receive an implant device, the instrument comprising:
a handle;
a support having an elongated body, the support mounted to the handle and extending in a longitudinal direction;
a drive shaft attached to the support and extending along the longitudinal direction;
a spacer mounted to a distal end of the support, the spacer dimensioned in the same shape as the implant, the spacer having a ramp-shaped member;
a stop to the support and movable along an axis parallel to the drive shaft, the stop configured to limit the depth at which the spacer is inserted into a body; and
a piercing member disposed on a distal end of the drive shaft, the drive shaft longitudinally displaceable with respect to the spacer, the piercing member configured to slide along the ramp-shaped member of the spacer so as to be driven beyond an outer surface of the spacer and pierce a bone located adjacent to the spacer.

12. The instrument as set forth in claim 11, wherein the support is a housing bounding a space, the drive shaft disposed within the housing and the piercing member is registered to slide along the ramp-shaped member of the spacer.

13. The instrument as set forth in claim 12, further including a sleeve mounted to the handle, wherein the sleeve is an elongated body configured to slidably house a portion of the drive shaft.

14. The instrument as set forth in claim 13, wherein the piercing member includes a first head and a second head, the first and second head are angled outwardly with respect to each other.

15. The instrument as set forth in claim 11, wherein the support is an elongated member extending axially from the handle and having a plurality of guides.

16. The instrument as set forth in claim 15, wherein the at least one of the plurality of guides is two guides, and each of the plurality of guides includes a ringed member and the drive shaft is slidingly disposed within the two guides and generally parallel to the support.

17. The instrument as set forth in claim 16, wherein the piercing member is pivotably mounted to the distal end of the drive shaft, and the end portion of the piercing member is registered to the ramp-shaped member of the spacer.
